SEQUENCE LISTING (6 sequences)

SEQ ID NO: 1 - DNA, 273 bp, synthetic construct
  source 1..273: mol_type = other DNA
cttaagtgttctgacgtctccaataaaatttctgcttgcttgtcttacttaaaacagggaggcgaggtgccagctgactgttgcacgggtgtaaaaggacttaacgacgcagccaagactacaccagatcgtcaaactgcctgtaattgtctcaagaccacattcaagagtaacaaagattttaaaagcgattttgccgcttctttgccctcgaaatgcggggtcaacatcccgtacaagatatcactggaaactgactgcaacaaggtaaaa

SEQ ID NO: 2 - protein, 91 aa, Artemisia vulgaris
LKCSDVSNKISACLSYLKQGGEVPADCCTGVKGLNDAAKTTPDRQTACNCLKTTFKSNKDFKSDFAASLPSKCGVNIPYKISLETDCNKVK

SEQ ID NO: 3 - protein, 89 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 4 - protein, 18 aa, synthetic construct
MFLQNLFLGFLAVVCANA

SEQ ID NO: 5 - protein, 24 aa, synthetic construct
MMKMMKFFCAMVVCMVVSSSYAEA

SEQ ID NO: 6 - protein, 26 aa, synthetic construct
MMAIKMMKVFCIMVVCMVVSTSYAES

(19) United States
(12) Patent Application Publication
(10) Pub. No.: US 2026/0216322 A1
(43) Pub. Date: Jul. 30, 2026

(54) RECOMBINANT ARTEMISIA VULGARIS POLLEN GROUP 3 ALLERGEN, AND PREPARATION METHOD THEREFOR AND USE THEREOF

(71) Applicant: ZONHONBIOPHARMA INSTITUTE INC., Jiangsu (CN)

(72) Inventors: Bruce Yong MA, Jiangsu (CN); Baixue ZHAO, Jiangsu (CN); Hui GAO, Jiangsu (CN); Yu ZHUANG, Jiangsu (CN)

(21) Appl. No.: 19/148,958

(22) PCT Filed: Jan. 12, 2024
(86) PCT No.: PCT/CN2024/072047

§ 371 Date: Jul. 17, 2025

(30) Foreign Application Priority Data
Jan. 17, 2023 (CN) 202310056355.1

(51) Int. Cl. A61K 39/36; A61P 37/04; C07K 1/18; C07K 1/34; C07K 14/415; C12N 15/81

(52) U.S. Cl. CPC A61K 39/36; A61P 37/04; C07K 1/18; C07K 1/34; C07K 14/415; C12N 15/815

(57) ABSTRACT

Provided are a recombinant Artemisia vulgaris pollen group 3 allergen protein Art v 3, an encoding gene thereof and an expression and purification method therefor. The amino acid sequence of the protein is shown in SEQ ID NO: 2, and the nucleotide sequence encoding the protein is shown in SEQ ID NO: 1. The protein can be used for preparing a medicament for the treatment of Artemisia pollen allergic diseases or a diagnostic reagent for the detection of the Artemisia pollen allergen.

### CROSS REFERENCE TO THE RELATED APPLICATIONS

This application is the national phase entry of International Application No. PCT/CN2024/072047, filed on Jan. 12, 2024, which is based upon and claims priority to Chinese Patent Application No. 202310056355.1, filed on Jan. 17, 2023. The entire contents of International Application No. PCT/CN2024/072047 and Chinese Patent Application No. 202310056355.1 are incorporated herein by reference.

### REFERENCE TO AN ELECTRONIC SEQUENCE LISTING

The instant application contains a Sequence Listing which has been submitted in XML format via EFS-Web and is hereby incorporated by reference in its entirety. Said XML copy is named SequenceListing.xml, created on 07/16/2025, and is 6,737 bytes in size.

### TECHNICAL FIELD

The present disclosure relates to the field of biopharmaceuticals, and more particularly to a recombinant Artemisia vulgaris pollen group 3 allergen, which possesses molecular characteristics and immunological activity equivalent to those of the native allergen. The present disclosure also relates to the method for preparation of the recombinant allergen and to its use.

### BACKGROUND

Pollen is one of the major triggers of seasonal allergies. Unlike food allergies, pollen allergy is transmitted through the air and is often difficult to avoid, leading to a range of allergic reactions such as rhinitis, dermatitis, and asthma, which seriously affect the quality of life of patients. In the United States, approximately 7% of adults and 9% of children suffer from pollen allergies (NIAID, National Institute of Allergy and Infectious Diseases), and the prevalence in Europe is estimated to be as high as 40% (G. D'Amato, 2007). In recent years, with the implementation of The Grain-for-Green Program and the expansion of green spaces in China, the incidence of pollen allergy has been increasing year by year, reaching up to 5% in high-prevalence areas.

Pollen from plants of the genus Artemisia is one of the important allergens responsible for pollinosis during the summer and autumn in China. According to a domestic study analyzing 215,210 allergen-specific IgE tests conducted between 2008 and 2010, Artemisia pollen showed the highest positivity rate among inhaled allergens. A 2015 study by Yang Qiongliang et al. indicated that Artemisia pollen is the most dominant sensitizing pollen in northern China.

The genus Artemisia (Artemisia L.) is one of the largest genera in the Asteraceae family in terms of species number, with more than 300 species distributed widely in the temperate, subtemperate, and subtropical regions of the Northern Hemisphere. Artemisia annua and Artemisia vulgaris are common pollen-sensitizing plants. Artemisia annua can be used to extract artemisinin, a highly effective antimalarial drug, and is one of the most common Artemisia species in China. It is also one of the earliest studied allergenic Artemisia plants in China. Artemisia vulgaris, also known as mugwort, is widely distributed in China, Mongolia, Russia, Europe, the United States, and Canada, and is one of the most deeply studied pollen-sensitizing plants. Other major allergenic Artemisia species include Artemisia sieversiana, Artemisia capillaris, Artemisia lavabdykufikua, Artemisia desertorum, and Artemisia argyi. The major allergenic proteins in pollens from different Artemisia species are Group 1 and Group 3 allergens. Group 3 allergens belong to the non-specific lipid transfer protein (ns-LTP) family, with a molecular weight of approximately 9 kDa. Group 3 allergens from different Artemisia species exhibit high homology, with sequence identity generally above 80%.

For allergic diseases, the World Health Organization (WHO) has proposed a Four-Pillar therapeutic strategy: avoidance of allergen exposure, symptomatic pharmacological treatment, allergen-specific immunotherapy (AIT), and education for physicians and patients. Among these, allergen avoidance refers to taking environmental control measures based on the identification of allergens, in order to minimize exposure. Effective allergen avoidance during the treatment of allergic diseases can not only reduce the incidence of allergic reactions in patients but also enhance the efficacy of pharmacological treatments and facilitate faster desensitization. Allergen-specific immunotherapy, also known as desensitization therapy, is a causal treatment and is the only therapy may affect the natural course of allergic diseases and change the immune response mechanism. It involves the administration of gradually increasing doses of allergens to patients to improve their tolerance to the allergen, thereby alleviating the symptoms triggered by allergen exposure, and ultimately achieving tolerance or even immune tolerance.

In 2021, a sublingual drop formulation containing Artemisia annua pollen allergen extract, developed by Zhejiang Wolwo Bio-Pharmaceutical Co., Ltd. for the treatment of Artemisia annua/mugwort pollen-induced allergic rhinitis, was approved for market authorization. The main component is an allergen protein extract derived from Artemisia annua pollen. The patent related thereto (CN101905022A) discloses that “Artemisia pollen is used as the raw material, which is defatted, extracted, and concentrated to produce the Artemisia pollen allergen extract.”

However, due to the limitations of raw material sources and manufacturing processes, natural allergen extracts inevitably exhibit quality problems, such as the presence of undefined nonallergenic materials, contaminants as well as high variabilities regarding contents and biological activity of individual allergens. (Valenta R, et al. Allergen Extracts for in vivo diagnosis and treatment of allergy: is there a future[J]. Journal of allergy & Clinical immunology in practice, 2018.). The 2018 EAACI Guidelines on Allergen Immunotherapy for Allergic Rhinoconjunctivitis, issued by the European Academy of Allergy and Clinical Immunology (EAACI), also pointed out that there are a number of potential drawbacks of mixing allergens, including a dilutional effect, potential allergen degradation due to enzymatic activity of some allergens and the difficulties of adequately demonstrating efficacy of a high number of allergen combinations. Standardized desensitization drugs approved by regulatory authorities such as EMA, HMA, and FDA have generally specified the inclusion of major allergenic proteins. For example, ODACTRA, used for dust mite allergy, contains the major allergenic proteins Der p 1, Der p 2, Der f 1, and Der f 2; GRAZAX, used for timothy grass allergy, contains the major allergenic protein Phl p 5; and RAGWIZAX, used for ragweed allergy, contains the major allergenic protein Amb a 1. On the other hand, the use of natural extracts for allergen diagnosis has limitations in sensitivity and specificity, and fails to precisely determine the patient's reactivity to specific allergenic components, which may lead to misdiagnosis.

Currently, no recombinant Artemisia pollen allergen protein drug has been approved for marketing or is undergoing clinical trials.

### SUMMARY

The applicant aims to provide a recombinant Artemisia vulgaris pollen allergen with a clearly defined major allergenic protein, in order to improve the controllability of product quality, ensure the therapeutic precision of allergen-specific immunotherapeutic drugs and the accuracy of allergen diagnostics for Artemisia pollen-induced allergic diseases, and lays a foundation for the druggability of recombinant Artemisia vulgaris pollen allergens.

An objective of the present disclosure is to provide a protein for the treatment of Artemisia pollen allergy, which is a recombinant Art v 3 protein. Art v 3 is an Artemisia vulgaris pollen group 3 allergen, and is a major allergenic protein belonging to the non-specific lipid transfer protein (ns-LTP) family, with a molecular weight of approximately 9 kDa. Studies have shown that group 3 allergen proteins from different Artemisia species exhibit high homology, with sequence identity generally above 80%. The recombinant Art v 3 protein has an amino acid sequence, disulfide bond pattern, and a molecular weight identical to those of the native Art v 3 protein, and has equivalent biological activity to that of the native Art v 3 protein.

Preferably, the amino acid sequence of the Art v 3 protein is shown in SEQ ID NO: 2.

The recombinant Art v 3 protein of the present disclosure has an amino acid sequence, a molecular weight, amino acid coverage, and disulfide bond pairing identical to those of the native Art v 3 protein, and has biological activity equivalent to that of the native Art v 3 protein. Compared to natural Artemisia vulgaris pollen extracts, the recombinant Art v 3 protein offers many advantages, such as avoiding batch-to-batch differences in the content and activity of major allergens resulting from variations in natural pollen sources, achieving more stable and controllable manufacturing processes and quality, preventing the degradation of major allergens caused by interactions with other components in natural pollen, and avoiding the elicitation of other allergic reactions. The recombinant protein meets the requirements for safety, efficacy, and controllable quality of modern biological products and can be used for the treatment and diagnosis of Artemisia pollen-induced allergic diseases, such as allergic rhinitis and asthma. It improves the precision of allergen-specific immunotherapy for Artemisia pollen allergy and the accuracy of Artemisia pollen allergen diagnosis, and has promising pharmaceutical potential.

Another objective of the present disclosure is to provide a DNA sequence encoding the Art v 3 protein, wherein the base sequence is set forth in SEQ ID NO: 1. The sequence has been codon-optimized for Pichia pastoris expression system, and more favorable for the expression of Art v 3 in Pichia pastoris.

Another objective of the present disclosure is to provide a design strategy of secretion signal peptides that facilitates the expression of the Art v 3 protein in the Pichia pastoris expression system. This signal peptide design not only increases the protein expression yield, but also ensures that the molecular characteristics of the recombinant Art v 3 protein are completely consistent with those of the native Art v3 protein. The signal peptides include: acid phosphatase signal peptide (Pho1), and natural signal peptide of Art v 3 protein (Natural signal 1, ns1). The amino acid sequence of the Pho1 signal peptide is shown in SEQ ID NO: 4, and the amino acid sequence of the ns1 signal peptide is shown in SEQ ID NO: 5. The inventors have found that the use of different secretion signal peptides to direct the secretion of Art v 3 in Pichia pastoris significantly affects the homogeneity and expression level of the recombinant Art v 3 protein. The preferred signal peptide Pho1 and ns1 are more favorable for the correct and efficient expression of Art v 3 in Pichia pastoris system. The resulting recombinant Art v 3 protein not only exhibits increased expression level, but also has a primary structure, molecular weight, an amino acid coverage, and disulfide bond pairing pattern identical to those of the native Art v 3.

Another objective of the present disclosure is to provide a vector comprising the codon-optimized Art v 3 gene described herein, wherein the vector includes strong promoter elements required for transcription of the target gene, such as the alcohol oxidase promoter (AOX promoter) or the glyceraldehyde-3-phosphate dehydrogenase promoter (GAP promoter). The vector is preferably selected from pAO815, pPIC9, pPIC9K, pPIC3.5, pPIC3.5K, pPICZ A, pPICZ B, pPICZ C, pGAPZ A, pGAPZ B, pGAPZ C, pPICZα A, pPICZα B, pPICZα C, pGAPZα A, pGAPZα B, and pGAPZα C.

Another objective of the present disclosure is to provide a Pichia pastoris strain comprising the above-described recombinant expression vector. Preferably, the Pichia pastoris strain is SMD1168, GS115, KM71, X33, or KM71H.

The recombinant Art v 3 protein-encoding gene of the present disclosure is more favorable for expression in Pichia pastoris. The inventors have discovered that recombinant expression strains constructed with different secretion signal peptide-vector combinations in Pichia pastoris exhibit significant differences in molecular weight consistency, expression yield, and correctness of the primary structure of the resulting Art v 3 protein. Through comparative analysis of multiple combinations, the inventors ultimately identified secretion signal-peptide vector combinations that enable the production of Art v 3 with a primary structure, molecular weight, amino acid sequence coverage, and disulfide bond pairing pattern completely identical to those of the native Art v 3 protein. The recombinant Art v 3 protein achieves an expression level of ≥60 mg/L and exhibits an amino acid sequence, disulfide bonds, and a molecular weight identical to those of the native Art v 3 protein. Furthermore, the recombinant Art v 3 protein demonstrates in vitro immunoreactivity to specific antibodies in the sera of allergic patients that is equivalent to that of the native Art v 3 protein.

Another objective of the present disclosure is to provide a method of expressing Art v 3 protein, comprising the following steps:

A. Construct a vector comprising the Art v 3 encoding gene described above:

The pPICZ and pGAPZ vector series lack a signal peptide within the exogenous gene expression cassette, thus allowing construction of an Art v 3 expression cassette containing either the natural signal peptide of Art v 3 or an artificially designed signal peptide such as Pho1. Specifically, the artificially synthesized Art v 3 sequence containing the natural signal peptide of Art v 3 (designed to include a start codon and a stop codon) is cloned into the multiple cloning site of the corresponding vector (for example, EcoRI and NotI), such that the open reading frame is positioned downstream of the promoter and upstream of the terminator, thereby constructing the recombinant expression vector.

The pPICZα and pGAPZα vector series contain an α-Factor signal peptide and signal peptide cleavage sites: Kex2 (amino acid sequence is KR) and Ste13 (amino acid sequence is EAEA) within the exogenous gene expression cassette. The gene encoding the Art v 3 protein can be cloned downstream of the Kex2 sequence (in this design, the α-Factor signal peptide is cleaved by Kex2 protease), or downstream of the Ste13 site (in this design, the α-Factor signal peptide can be cleaved by Kex2 and/or Ste13 proteases). Taking the pPICZα A vector as an example, cloning the Art v 3 gene downstream of the Kex2 site can be achieved by cloning the target gene between the XhoI and NotI sites; cloning the Art v 3 gene downstream of the Ste13 site can be achieved by cloning the target gene between the EcoRI and NotI sites. By way of example, a recombinant expression vector for secretory expression of Art v 3 using the α-Factor signal peptide was constructed.

B. Linearize the vector obtained in step A, then transform the vector into a Pichia pastoris strain, and culture the transformed strain under appropriate conditions.

C. Recover and purify the protein expressed by culturing in step B.

The vectors are preferably selected from pPICZ A, or pGAPZ A.

The Pichia pastoris strains are preferably selected from KM71 or X33 strains.

Another objective of the present disclosure is to provide a purification method of recombinant Art v 3 protein, comprising the following steps:

A. Centrifuge the Art v 3 fermentation broth at low temperature and high speed to collect the supernatant; concentrate the supernatant by ultrafiltration using a 3 kDa cutoff membrane; perform buffer exchange with 10 mM PB, pH 7.0; and filter through a 0.45 m membrane.

B. Perform cation exchange chromatography: equilibrate a chromatography column with equilibration buffer; pass the fermentation broth obtained in step A through a chromatographic resin using a purification system; elute with a gradient of elution buffer and collect the elution peaks. The equilibration buffer is 10 mM acetic acid-sodium acetate, pH 5.0; the elution buffer is 10 mM acetic acid-sodium acetate, 1.0 M NaCl, pH 7.0.

C. Dilute the Art v 3 protein peak collected in step B with equilibration buffer; equilibrate a chromatography column with equilibration buffer; load the diluted Art v 3 protein solution onto a hydrophobic interaction chromatography resin; and collect the elution peak. The equilibration buffer is 2.5 M (NH₄)₂SO₄, 10 mM PB, pH 7.4; the elution buffer is 10 mM PB, pH 7.4.

D. Subject the target protein peak collected in step C to ultrafiltration and buffer exchange; the buffer is 10 mM citric acid-sodium citrate (pH 5.0); perform sterile filtration to obtain the Art v 3 protein stock solution.

After optimization of the fermentation and cultivation process and purification methods, the recombinant Art v 3 produced by this disclosure meets the requirements for recombinant DNA products for human use in multiple aspects including purity, process-related impurities, and molecular characterization. The HPLC-SEC and HPLC-RP purity are ≥99%, expression yield reaches ≥60 mg/L, and scale-up production yield is >0.2 g/L. The recombinant protein possesses an amino acid sequence, disulfide bonds, and a molecular weight identical to those of the native Art v 3 protein, and demonstrates immunoreactivity equivalent to the native Art v 3 protein with specific antibodies in sera from allergic patients in vitro, showing promising pharmaceutical potential.

Compared to natural allergen extracts, the recombinant Art v 3 protein of the present disclosure has many advantages, such as avoiding batch-to-batch differences in the content and activity of major allergens resulting from variations in natural pollen sources, achieving more stable and controllable allergen manufacturing processes and quality, preventing the degradation of major allergens caused by interactions with other components in natural pollen, and avoiding the elicitation of other allergic reactions. The recombinant Art v 3 protein of the present disclosure meets the requirements for safety, efficacy, and controllable quality of modern biological products. The recombinant Art v 3 protein can be used in diagnostics to identify the allergenic molecules that truly cause allergic reactions and to reveal cross-reactivity. It can also be used in desensitization immunotherapy, exhibiting good tolerance, safety, and efficacy, ensuring precision in desensitization treatment of Artemisia pollen allergy and accuracy in allergen diagnosis, and lays a foundation for the druggability of recombinant Artemisia pollen allergens.

### BRIEF DESCRIPTION OF THE DRAWINGS

FIG. 1 is the electrophoretic analysis of culture supernatants after shake-flask expression of four strains of different construct design. Lanes 1-4 correspond to the supernatants from strains X33-pGAPZα A-Art v 3 (signal peptide: α-Factor), X33-pGAPZ A-Art v 3 (signal peptide: ns1), X33-pGAPZ A-Art v 3 (signal peptide: ns2), and X33-pGAPZ A-Art v 3 (signal peptide: Pho1).

FIG. 2-1 is a fragment coverage map of the Art v 3 sample showing disulfide bonds involving ~C27, ~C28/C13/C73.

FIG. 2-2 is a fragment coverage map of the Art v 3 sample showing disulfide bonds involving ~~C48, ~C50/C3/C87.

FIG. 3 is the result of molecular weight determination of Art v 3.

FIG. 4 is a chromatogram showing the purity analysis of purified recombinant Art v 3 by HPLC-SEC.

FIG. 5 is a chromatogram showing the purity analysis of purified recombinant Art v 3 by HPLC-RP.

FIG. 6 is the electrophoretic analysis of the fermentation broth supernatant from the scaled-up production of recombinant Art v 3. Lane 1 shows the SDS-PAGE analysis result under reducing conditions of the fermentation supernatant.

FIG. 7 is a graph showing the IgE-binding inhibition curves of the recombinant and native Art v 3 protein in positive sera.

### DETAILED DESCRIPTION OF THE EMBODIMENTS

The present disclosure is further described with reference to specific embodiments. It should be understood that the embodiments are provided for illustrative purposes only and do not limit the scope thereof.

### Example 1: Codon Optimization of the Art v 3 Gene

Based on the publicly available Art v 3 protein sequence (GenBank accession number: ACE07186), as shown in SEQ ID NO: 2, the inventors performed reverse translation to obtain the DNA sequence encoding the protein and subsequently carried out codon optimization on the coding DNA sequence. The final optimized gene sequence is shown in SEQ ID NO: 1. The codon-optimized DNA sequence was synthesized by Nanjing GenScript Biotech Co., Ltd. and supplied in the form of a subcloned plasmid lyophilized powder.

### Example 2: Construction of Recombinant Art v 3 Expression Strains

In this example, various recombinant Art v 3 expression strains were constructed using plasmids pAO815, pPIC9, pPIC9K, pPIC3.5, pPIC3.5K, pPICZ A, pPICZ B, pPICZ C, pGAPZ A, pGAPZ B, pGAPZ C, pPICZα A, pPICZα B, pPICZα C, pGAPZα A, pGAPZα B, or pGAPZα C, and host strains SMD1168, GS115, KM71, X33, or KM71H, in combination with multiple signal peptides. To illustrate the construction process, Art v 3 recombinant expression strains were constructed using plasmids pGAPZα A, pPICZα A, pGAPZ A, and pPICZ A, in combination with signal peptides α-Factor, Pho1, or natural signal peptides ns1 and ns2. Other construct forms are not described in detail herein.

1. Construction of Art v 3 Expression Constructs with Different Signal Peptides

1.1 Design of α-Factor-Art v 3 construct: Primers were designed based on the codon-optimized Art v 3 gene from Example 1, introducing appropriate restriction enzyme cleavage sites. The Art v 3 DNA sequence (including the stop codon) was amplified by PCR and cloned between the XhoI and NotI restriction sites in the multiple cloning site of pGAPZα A or pPICZα A plasmids via XhoI and NotI restriction sites, generating recombinant expression plasmids. Thus, the Art v 3 gene was positioned immediately downstream of the gene of α-Factor signal peptide (amino acid sequence shown in SEQ ID NO: 3) in the plasmid, enabling fusion expression.

1.2 Design of Pho1-Art v 3 construct: Primers were designed based on the codon-optimized Art v 3 gene from Example 1. Overlapping sequences corresponding to the vector insertion sites were added to both ends of the gene, and a start codon as well as the DNA sequence encoding the Pho1 signal peptide (amino acid sequence shown in SEQ ID NO: 4) were introduced at the 5′ end of the gene. The Pho1-Art v 3 DNA sequence was amplified by PCR and seamlessly cloned into predetermined sites of pGAPZ A or pPICZ A plasmids, generating recombinant expression plasmids. In the recombinant expression plasmids, the Pho1-Art v 3 gene was positioned downstream of the core promoter of the plasmid (e.g., GAP promoter as the core promoter of pGAPZ A, and AOX1 promoter as the core promoter of pPICZ A, hereinafter the same) and upstream of the terminator (AOX1 terminator, hereinafter the same), forming an open reading frame.

1.3 Design of natural signal peptide-Art v 3 construct (Natural signal type Art v 3, nsArt v 3): Two natural signal peptides investigated in the present disclosure are denoted as ns1 and ns2, with amino acid sequences shown in SEQ ID NO: 5 and SEQ ID NO: 6, respectively. Primers were designed based on the codon-optimized Art v 3 gene from Example 1. Overlapping sequences corresponding to the vector insertion sites were added to both ends of the gene, and a start codon as well as the DNA sequence encoding the corresponding natural signal peptide were introduced at the 5′ end of the gene. The ns1-Art v 3 and ns2-Art v 3 DNA sequences were amplified by PCR and seamlessly cloned into predetermined sites of pGAPZ A or pPICZ A plasmids, respectively, generating the corresponding recombinant expression plasmids. In the recombinant expression plasmids, the ns1-Art v 3 or the ns2-Art v 3 gene was positioned downstream of the core promoter and upstream of the terminator, forming an open reading frame.

### 2. Construction of Recombinant Expression Strains

The recombinant expression plasmids constructed in step 1 were transformed into Escherichia coli cloning hosts, such as DH5a or Top10. After cultivation, plasmids were extracted and sequenced to confirm the correct construction of the recombinant plasmids, generating subcloning strains. The subcloning strains were cultured for large-scale plasmid extraction to obtain high-concentration recombinant expression plasmids. The recombinant expression plasmids were linearized with appropriate restriction endonucleases, and the linearized fragments were purified and recovered. The purified linearized DNA was then transformed into appropriate competent Pichia pastoris cells, such as strains SMD1168, GS115, KM71, X33 or KM71H, via electroporation followed by plating onto plates for cultivation.

### 3. Screening of Expression Strains

Single colonies were picked from the transformation plates obtained in step 2 and inoculated onto plates containing appropriate antibiotics (for the above vectors, bleomycin was used). After cultivation for an appropriate period, selection pressure was applied using different antibiotic concentrations. Single colonies from plates with high antibiotic concentrations were finally selected, cultured, and analyzed for expression, leading to the identification of high-expression strains.

### 4. Expression and Identification

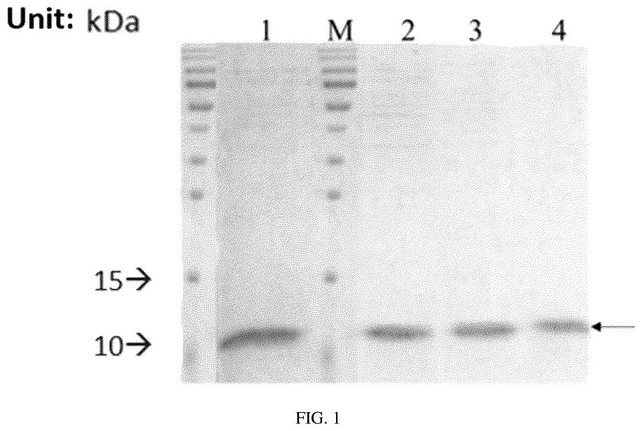

Expression strains harboring constitutive expression vectors based on the GAP promoter, such as pGAPZα A or pGAPZ A, were cultured in YPD liquid medium with shaking for 72 hours. Equal volumes of culture supernatant were collected for SDS-PAGE or Dot blot analysis. Expression strains harboring inducible expression vectors based on the AOX1 promoter, such as pPICZα A or pPICZ A, were cultured in YPD liquid medium with shaking for 24 hours, followed by induction with 0.5% methanol (v/v) added to the culture (supplemented once every 24 hours). After 72 hours of inducible expression, equal volumes of culture supernatant were collected for SDS-PAGE or Dot blot analysis. Strains exhibiting higher expression levels were selected for subsequent 3-liter scale fermenter studies to compare fermentation expression yields, as summarized in Table 1. The SDS-PAGE analysis results of expression identification are shown in FIG. 1.

### Example 3: Purification of Recombinant Art v 3 Protein

A. The fermentation broth of Art v 3 was subjected to low-temperature and high-speed centrifugation to collect the supernatant, which was then concentrated by ultrafiltration using a 3 kDa cutoff membrane. The buffer was exchanged with 10 mM PB, pH 7.0, followed by filtration through a 0.45 m membrane.

B. Perform cation exchange chromatography: A chromatography column was equilibrated with equilibration buffer; then, the fermentation broth obtained in step A was passed through a chromatography resin using a purification system; subsequently, elution was performed with a gradient of elution buffer, and elution peaks were collected. The equilibration buffer was 10 mM acetic acid-sodium acetate, pH 5.0; the elution buffer was 10 mM acetic acid-sodium acetate, 1.0 M NaCl, pH 7.0.

C. The Art v 3 protein peak collected in step B was diluted with equilibration buffer; and a chromatography column was equilibrated with equilibration buffer; the diluted Art v 3 protein solution was loaded onto a hydrophobic interaction chromatography resin, and elution peaks were collected. The equilibration buffer was 2.5 M (NH₄)₂SO₄, 10 mM PB, pH 7.4; the elution buffer was 10 mM PB, pH 7.4.

D. The target protein peak collected in step C was subjected to ultrafiltration and buffer exchange with 10 mM citric acid-sodium citrate buffer, pH 5.0, followed by sterile filtration to obtain the Art v 3 protein stock solution.

### Example 4: Evaluation of the Effect of Different Signal Peptide Based Constructs on Art v 3 Expression

1. The purified products prepared in Example 3 were analyzed by LC-MS to determine their molecular weights. The molecular weight of the purified Art v 3 protein was assessed for correctness based on the LC-MS results.

2. For Art v 3 products with correct or incorrect molecular weights, further analysis of the amino acid sequences was performed. The results are summarized in the table below.

TABLE 1
Expression levels and characteristics of Art v 3 expressed
by different signal peptide- vector combinations

| | | | | | | Amino | |
|---|---|---|---|---|---|---|---|
| | | Secretion signal | Expression | | Molecular | acid | Signal peptide |
| Host | Vector | peptide | level | | weight | coverage | residue |
| X33 | pGAPZα | α-Factor | 100 | mg/L | incorrect, | 100% | residual signal |
| | A | (The target gene | | | larger than | | peptide sequence |
| | | was positioned | | | the | | EEGVSLEKR |
| | | downstream of | | | theoretical | | was covered |
| | | Kex2 site) | | | value | | |
| | pGAPZ | Pho1 | 50 | mg/L | correct | 100% | no residual signal |
| | A | | | | | | peptide sequence |
| | | natural signal | 60 | mg/L | correct | 100% | no residual signal |
| | | peptide 1 (ns1) | | | | | peptide sequence |
| | | natural signal | 45 | mg/L | incorrect, | 100% | residual signal |

| | | |
|---|---|---|
| peptide 1 (ns2) | larger than | peptide sequence |
| | the | ES was covered |
| | theoretical | |
| | value | |

In this example, the recombinant expression performance of Art v 3 using various signal peptides was compared, and the effect of different signal peptide choices on the molecular correctness of the final Art v 3 protein product was evaluated. It was found that the acid phosphatase signal peptide (Pho1) and the natural signal peptide (ns1) were capable of directing the correct and efficient expression of Art v 3 in Pichia pastoris, resulting in high expression levels of the recombinant Art v 3 protein, which exhibited molecular characteristics fully consistent with those of native Art v 3. In contrast, other signal peptide-vector combinations, such as pGAPZα A-α-Factor and pGAPZ A-ns2 (as listed in Table 1), failed to achieve correct expression or resulted in low expression levels.

The acid phosphatase signal peptide Pho1 and the natural signal peptide ns1 were both completely cleaved upon secretion. The recombinant Art v 3 protein produced using the two signal peptides exhibited correct molecular weights and amino acid sequences identical to those of the native Art v 3, and the corresponding expression strains yielded high levels of the target protein. As shown in Examples 5 and 8, the recombinant Art v 3 protein produced using the two signal peptides exhibited correct disulfide bond pairing and demonstrated in vitro biological activity equivalent to that of the native Art v 3 protein. As shown by the expression results of pGAPZ A-Art v 3 constructs with different signal peptides listed in Table 1, the recombinant expression strains constructed with the signal peptides Pho1 and ns1 yielded target protein with correct molecular characteristics and high expression levels. In contrast, signal peptides such as α-Factor and the natural signal peptide ns2 were not completely cleaved after the secretion of the target protein, resulting in recombinant Art v 3 protein with molecular weight and amino acid sequence that were inconsistent with those of the native Art v 3 protein. The residual signal peptide amino acids may lead to reduced biological activity, increased immunogenicity of the target protein, and other negative effects.

### Example 5: Molecular Weight and Disulfide Bond Analysis of Art v 3 Protein

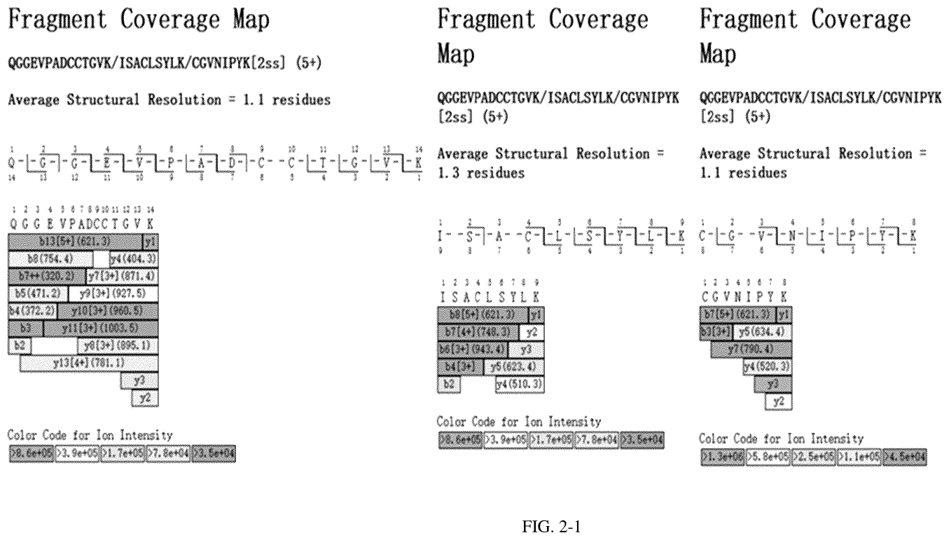

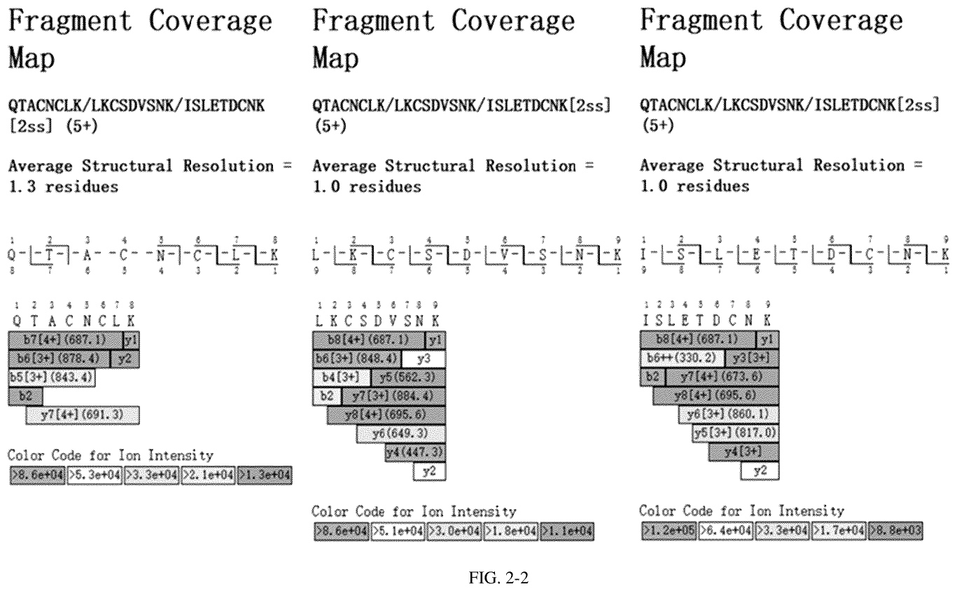

Correct pairing of disulfide bonds is critical for maintaining the higher-order structure and activity of proteins and other biomacromolecules. The recombinant Art v 3 protein with correct molecular weight, expressed from the Pho1-Art v 3 and ns1-Art v 3 constructs, was subjected to molecular weight and disulfide bond analysis using the Thermo Scientific Q Exactive LC-MS system. Four theoretical disulfide bonds were identified in the recombinant Art v 3 protein: C3~C50, C13~C27, C28~C73, and C48~C87, which are consistent with those reported in the database. The measured molecular weights were identical to the theoretical values. Taking the recombinant Art v 3 protein expressed by the recombinant expression strain constructed using X33-pGAPZ A with the ns1 signal peptide as an example, the corresponding results are shown in FIG. 2 and FIG. 3.

### Example 6: Purity Analysis of Recombinant Art v 3 Protein

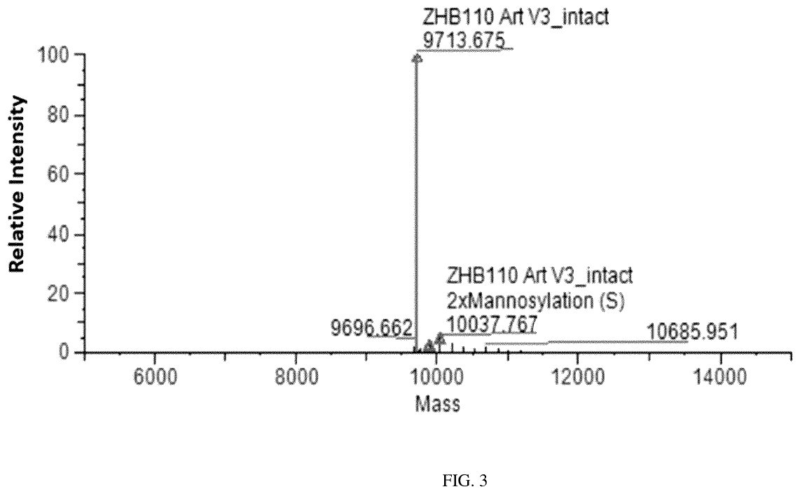

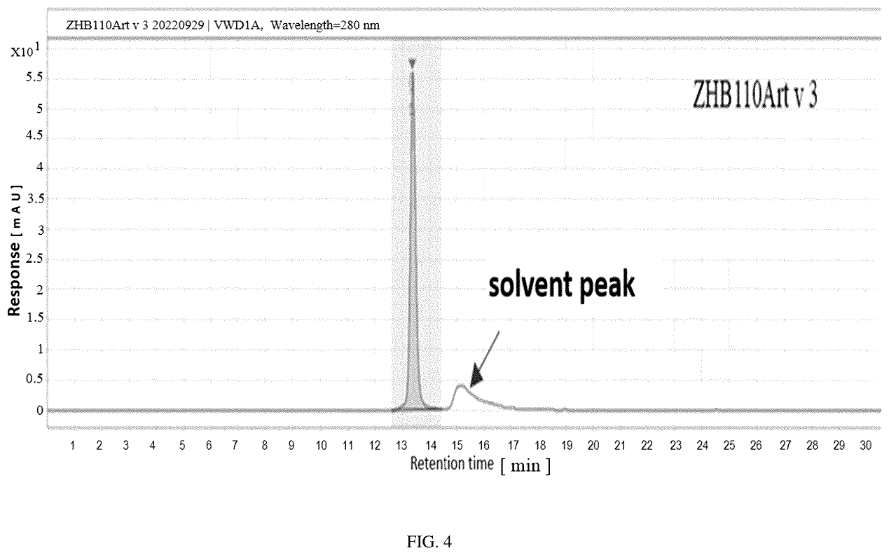

Taking the recombinant Art v 3 protein expressed by the recombinant strain constructed using X33-pGAPZ A with the ns1 signal peptide in Example 3 as an example. The highly purified Art v 3 protein was analyzed by HPLC-SEC and HPLC-RP, demonstrating purity of 99-100% or higher, meeting the requirements for pharmaceutical-grade purity. Chromatograms of the recombinant Art v 3 expressed with the ns1 signal peptide, shown in FIG. 4 and FIG. 5, reveal main peak purities of 100% and 99.80%, respectively, based on peak integration.

### Example 7: Purification of Native Art v 3 Protein

1. Preparation of Crude Extract: Defatted Artemisia vulgaris pollen (purchased from Stallergenes Greer) was weighed. A 50 mM PB solution at pH 7.0 was prepared and added to the pollen at a ratio of 1:10 (w/v). Extraction was carried out at 2-8° C. for 48-72 hours. The mixture was centrifuged at 4000 rpm at low temperature, and the supernatant was collected as the crude extract.

2. Chromatographic Purification: The crude extract obtained in step 1 was purified according to the purification procedure described in Example 3. After purification, electrophoresis analysis was performed, and fractions exhibiting satisfactory purity were collected for subsequent procedures.

3. Ultrafiltration and Buffer Exchange: The target fractions collected in step 2 were subjected to ultrafiltration and buffer exchange using PBS (pH 7.4); the resulting solution was stored frozen at −20° C. or below until use.

### Example 8: Scale-up of Fermentation and Purification Processes for Recombinant Art v 3 Strains

In this example, the recombinant expression strain constructed using X33-pGAPZ A with the ns1 signal peptide was used to scale up the fermentation and purification processes for recombinant Art v 3. The expression level and purification yield were further improved, and other preferred constructs of the disclosure can also be well scaled up for production.

1. The genetically engineered strain constructed in Example 2 was streaked from glycerol stock onto YPD solid agar medium and incubated at a constant temperature of 30° C. for 48 to 72 hours until single colonies formed, completing strain activation.

2. A single colony from step 1 was inoculated into YPD primary seed medium and cultured at 30° C. with shaking at 220 rpm for 12 to 16 hours. The final OD₆₀₀ was between 1 and 4, constituting the primary seed culture.

3. The primary seed culture from Step 2 was inoculated at 1% (v/v) into YPD secondary seed medium and cultured at 30° C. with shaking at 220 rpm for 18 to 24 hours. The final OD₆₀₀ was between 8 and 15, constituting the secondary seed culture.

4. The secondary seed culture from step 3 was inoculated at 1% (v/v) into a seed fermenter containing YPD medium for cultivation. Seed fermenter cultivation conditions were as follows: pressure 0.2-0.5 bar, temperature 28-30° C., and dissolved oxygen (DO) controlled between 15% and 35% by adjusting the agitation speed and the aeration rate of the fermenter. Cultivation was terminated when the OD₆₀₀ reached between 8 and 15. The seed fermenter volume was 30 L.

5. The seed fermenter culture broth from step 4 was transferred to the production fermenter for cultivation. Production fermenter cultivation conditions were as follows: pressure 0.2-0.5 bar, temperature 28-30° C., dissolved oxygen (DO) controlled between 15% and 35% by adjusting the agitation speed and aeration rate of the fermenter. The production fermenter culture medium was BSM inorganic medium. Fed-batch cultivation was performed. After the carbon source (glycerol) in the initial medium of the production fermenter was depleted, the second feeding stage was initiated using 50% glycerol or glucose as feed carbon source, with a feed rate of 5-15 mL/L/h. Fed-batch cultivation was continued for 24 to 36 hours, after which fermentation was concluded. The initial fermentation volume in the production fermenter after inoculation was 300 L.

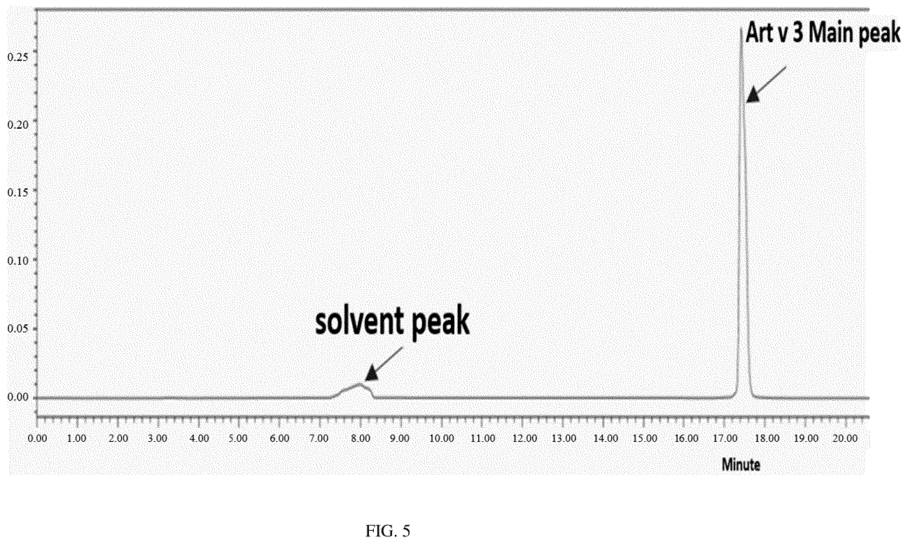

6. At the end of fermentation, the broth was centrifuged using a continuous-flow centrifuge to collect the supernatant. The supernatant was then filtered through a 0.45 μm cutoff filter to remove impurities. The fermentation supernatant was analyzed by electrophoresis, with results shown in FIG. 6. The target protein yield in the fermentation supernatant reached ≥0.2 g/L. The fermentation supernatant was purified according to the procedure described in Example 3. Purity of the final purified product, as determined by both HPLC-SEC and HPLC-RP, reached ≥99%, meeting pharmaceutical-grade standards. A single batch yielded the purified product of Art v 3 protein≥20 g, demonstrating excellent feasibility for process scale-up and laying the foundation for future production.

### Example 9: Activity Assay of Art v 3 Protein

In this example, the recombinant Art v 3 protein expressed by the recombinant strain constructed using X33-pGAPZ A with ns1 signal peptide was used to demonstrate that the recombinant Art v 3 protein of the present disclosure exhibits equivalent activity to that of the native Art v 3 protein. The recombinant Art v 3 protein expressed via other preferred constructs of the present disclosure also exhibits activity equivalent to that of the native Art v 3 protein.

1. Highly purified rArt v 3 from Example 3 and native Art v 3 protein from Example 7 were each diluted with 50 mM NaH₂PO₄ buffer (pH 7.2) to 0.5 μg/mL, 100 μL per well was added, and the plates were coated overnight at 4° C.

2. Sample preparation: The recombinant or native protein was diluted with blocking solution (2% BSA/PBST) to an initial concentration of 500 μg/mL (S1), followed by a 4-fold serial dilution to obtain 10 gradients (S2-S11). Each diluted sample was mixed 1:1 with positive serum (diluted 1:15), and the mixtures were incubated overnight at 4° C.

3. The following day, the ELISA plate was taken out and washed 4 times with PBST, then 200 μL of 2% BSA/PBST was added per well for blocking at 37° C. for 2 hours.

4. After blocking, the blocking solution was discarded. The mixed and incubated samples from the previous step were added at 100 μL per well, and incubated at 37° C. for 1.5 hours at 300 rpm.

5. The plate was washed 3 times with PBST, then 100 μL of 1:1500 diluted mouse anti-human IgE-HRP secondary antibody was added to each well, followed by incubation at 37° C. for 1 hour at 300 rpm.

6. The plate was washed 3 times with PBST, then 100 μL of TMB VII chromogenic solution was added per well. After reacting at 37° C. for 10 minutes, 50 μL of stop solution (2M H₂SO₄) was added to each well, and OD₄₅₀nm was measured immediately.

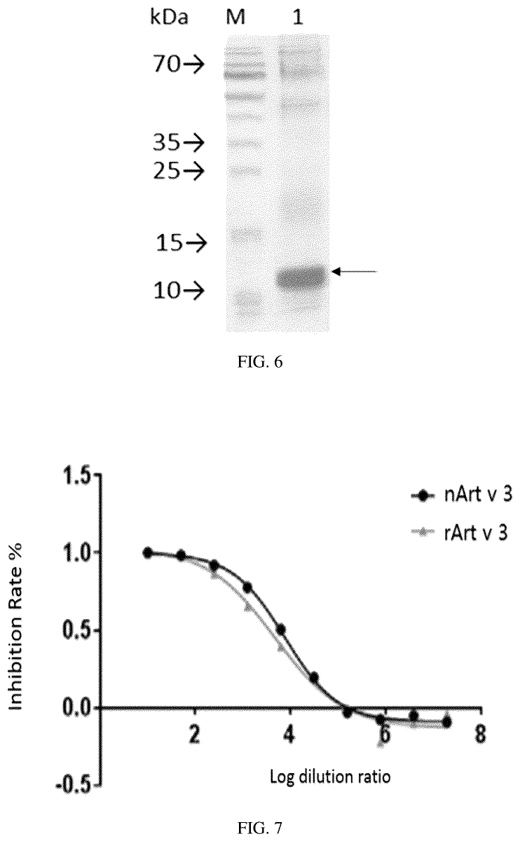

7. Results analysis: As shown in FIG. 7, the IgE-binding inhibition curves in positive serum were essentially identical between the recombinant Art v 3 protein of the present disclosure and the native Art v 3 protein. Using the native Art v 3 protein as the reference standard, the recombinant Art v 3 protein exhibited equivalent performance, indicating that the recombinant Art v 3 protein possesses in vitro immunoreactivity to the allergen-specific IgE antibody in the sera of allergic patients equivalent to that of the native Art v 3 protein.

1. A protein for treating Artemisia pollen allergy, wherein the protein is a recombinant Artemisia vulgaris pollen group 3 allergen (Art v 3), the recombinant Art v 3 has an amino acid sequence, a disulfide bond, and a molecular weight identical to a native Art v 3 protein, and possesses an immunoreactivity in vitro to allergen-specific antibodies in a serum of an allergic patient equivalent to the immunoreactivity of the native Art v 3 protein.

2. The protein for treating Artemisia pollen allergy according to claim 1, wherein the amino acid sequence is set forth in SEQ ID NO: 2.

3. A polynucleotide encoding the protein for treating Artemisia pollen allergy according to claim 2, wherein the polynucleotide has a base sequence as set forth in SEQ ID NO: 1.

4. A vector comprising the polynucleotide encoding the protein for treating Artemisia pollen allergy according to claim 3, wherein the vector is selected from the group consisting of pAO815, pPIC9, pPIC9K, pPIC3.5, pPIC3.5K, pPICZ A, pPICZ B, pPICZ C, pGAPZ A, pGAPZ B, pGAPZ C, pPICZα A, pPICZα B, pPICZα C, pGAPZα A, pGAPZα B, and pGAPZα C.

5. A Pichia pastoris strain comprising the vector according to claim 4, wherein the Pichia pastoris strain is selected from the group consisting of SMD1168, GS115, KM71, X33, and KM71H.

6. A method of expressing the protein for treating Artemisia pollen allergy according to claim 1, comprising steps of:
step A: constructing a vector comprising a gene encoding Art v 3, wherein the vector comprises a polynucleotide encoding the protein for treating Artemisia pollen allergy, the polynucleotide has a base sequence as set forth in SEQ ID NO: 1, the recombinant Art v 3 has the amino acid sequence as set forth in SEQ ID NO: 2, and the vector is selected from the group consisting of pAO815, pPIC9, pPIC9K, pPIC3.5, pPIC3.5K, pPICZ A, pPICZ B, pPICZ C, pGAPZ A, pGAPZ B, pGAPZ C, pPICZα A, pPICZα B, pPICZα C, pGAPZα A, pGAPZα B, and pGAPZα C;
step B: linearizing the vector of the step A, and transforming the vector into a Pichia pastoris strain, followed by culturing under appropriate conditions; and
step C: recovering and purifying the protein expressed by culturing in the step B.

7. A method of purifying the protein for treating Artemisia pollen allergy according to claim 1, comprising steps of:
step A: centrifuging an Art v 3 fermentation broth at low temperature and high speed to collect a supernatant; concentrating the supernatant by ultrafiltration using a 3 kDa cutoff membrane; performing buffer exchange with 10 mM PB (pH 7.0); and filtering through a 0.45 μm membrane;
step B: performing cation exchange chromatography; equilibrating a chromatography column with equilibration buffer; passing the Art v 3 fermentation broth obtained in the step A through a chromatographic resin using a purification system; eluting with a gradient of elution buffer and collecting elution peaks; wherein the equilibration buffer is 10 mM acetic acid-sodium acetate, pH 5.0, and the elution buffer is 10 mM acetic acid-sodium acetate, 1.0 M NaCl, pH 7.0;
step C: diluting an Art v 3 protein peak collected in the step B with the equilibration buffer; equilibrating a second chromatography column with a second equilibration buffer; loading the diluted Art v 3 protein solution onto a hydrophobic interaction chromatography resin; collecting the elution peaks; wherein the second equilibration buffer is 2.5 M (NH₄)₂SO₄, 10 mM PB, pH 7.4, and the elution buffer is 10 mM PB, pH 7.4; and
step D. subjecting a target protein peak collected in the step C to ultrafiltration and a second buffer exchange; wherein a buffer used in the second buffer exchange is 10 mM citric acid-sodium citrate (pH 5.0); performing sterile filtration to obtain an Art v 3 protein stock solution.

8. A method for treating Artemisia pollen allergic diseases, comprising administering to a subject a medicament comprising the protein according to claim 1 in an effective amount.

9. A method for detecting Artemisia pollen allergens, comprising administering to a subject a diagnostic reagent comprising the protein according to claim 1 in an effective amount.

10. The method of purifying the protein for treating Artemisia pollen allergy according to claim 7, wherein the amino acid sequence of the recombinant Art v 3 is set forth in SEQ ID NO: 2.

11. The method according to claim 8, wherein the amino acid sequence of the recombinant Art v 3 is set forth in SEQ ID NO: 2.

12. The method according to claim 9, wherein the amino acid sequence of the recombinant Art v 3 is set forth in SEQ ID NO: 2.
